(19) United States
(12) Patent Application Publication
(10) Pub. No.: US 2008/0003260 A1
(43) Pub. Date: Jan. 3, 2008

(54) Use of 5-fluorourcil, delivered by iontophoresis as an inhibitor of cell proliferation in the eye, bulbar and palpebral conjunctiva, eyelid, peri-orbital soft tissues and skin

(75) Inventors: Stephen Warren, Salt Lake City, UT (US); Steven Hamilton, Salt Lake City, UT (US)

Correspondence Address: FACTOR & LAKE, LTD, 1327 W. WASHINGTON BLVD., SUITE 5G/H, CHICAGO, IL, 60607, US

(73) Assignee: IOMED, INC., Salt Lake City, UT (US)

(21) Appl. No.: 11/900,170

(22) Filed: Sep. 10, 2007

Related U.S. Application Data
(62) Division of application No. 10/707,794, filed on Jan. 13, 2004.

(51) Int. Cl. A61K 31/513; A61K 9/00; A61P 27/02

(52) U.S. Cl. 424/427; 514/274

(57) ABSTRACT

A method for treating neoplastic, angiogenic, fibroblastic, and/or immunosuppressive ocular irregularities of a living subject, comprising the steps of: providing a living subject, wherein the living subject includes an affected ocular area having a neoplastic, angiogenic, fibroblastic, and/or immunosuppressive irregularity; providing a 5′ fluorouracil based medicament, wherein the 5′ fluorouracil based medicament is capable of inhibiting DNA synthesis; associating a therapeutically effective concentration of the 5′ fluorouracil based medicament with the affected ocular area of the living subject; and decreasing the neoplastic, angiogenic, fibroblastic, and/or immunosuppressive ocular irregularity of the living subject.

### CROSS-REFERENCE TO RELATED APPLICATIONS

This application is a divisional application of pending U.S. application Ser. No. 10/707,794 filed on Jan. 13, 2004, which, is a national phase patent application under 35 U.S.C. §371 of International Application Number PCT/US02/22860, filed on Jul. 19, 2002, which claims priority to U.S. Provisional Application Ser. No. 60/306,788, filed on Jul. 20, 2001—all of these documents are incorporated herein by reference.

### BACKGROUND OF THE INVENTION

1. Field of the Invention

The present invention relates in general to methods for treating neoplastic, angiogenic, fibroblastic, and/or immunosuppressive ocular irregularities, and more particularly, to methods for treating the same via administration of one or more 5′ fluorouracil based medicament(s) which are capable of acting as an inhibitor of DNA synthesis, and blocking the proliferation of multiple types of neoplastic cells, including malignant and non-malignant lesions. The present invention further relates to the controlled administration of 5′ fluorouracil based medicaments to an affected area of a living subject's eye.

2. Background Art

5′ fluorouracil based medicaments have been known in the art for years, and have been shown to possess anti-neoplastic, anti-angiogenic, anti-fibroblastic, and/or immunosuppressive activities. While administering 5′ fluorouracil based medicaments have been identified as a promising remedy to treat many of the above-identified irregularities, delivering 5′ fluorouracil based medicaments to an affected area of a living subject's eye has remained heretofore largely problematic. Indeed, known prior art methods of administering 5′ fluorouracil based medicaments, identified hereinbelow, are replete with substantial drawbacks and/or life threatening complications.

For example, delivering 5′ fluorouracil based medicaments to an affected, local area of a living subject's eye using a systemic delivery method is problematic because of the many severe, sometimes life threatening, side effects associated with systemic delivery of 5′ fluorouracil based medicaments, such as, for examples, hepatitis, liver fibrosis, cirrhosis, leukopenia (bone marrow suppression), mucositis, ulcerative stomatitis, skin rash, nausea, abdominal distress, malaise, fatigue, chills and fever, diarrhea, gastrointestinal ulceration or perforation, pancreatitis, pericarditis, hypotension, deep venous thrombosis, thrombophlebitis, interstitial pneumonitis, headaches, drowsiness, cognitive dysfunction, reduced immunity, rash, photosensitivity, nephropathy, hematuria, alopecia, defective oogenesis, oligospermia, infertility, miscarriage, and birth defects.

Local delivery of 5′ fluorouracil based medicaments via interocular injection remains problematic because of the opportunity for, among other things, retinal detachment, bleeding into the interior of the eye, increased interocular pressure, and increased risk of secondary infection. Although perhaps justifiable for occasional acute conditions, these risk factors render interocular injection undesirable as a delivery mode for anything less than critically acute ocular irregularities. Furthermore, interocular injections can not only be scary and unpleasant, but also extremely painful for the patient.

In addition to the above-identified problems associated with interocular injection, peribular or subconjuctival injection of 5′ fluorouracil based medicaments can be problematic, because such injections may not deliver sufficient quantities to the interior of the eye. Moreover, peribular or subconjuctival injections are demanding of the physician inasmuch as placement of the needle requires an extremely high level of precision.

Topical administration of 5′ fluorouracil based medicaments to an affected, local area of a living subject's eye is problematic due to its ineffectiveness for many applications, including affected areas in the back of the eye.

### SUMMARY OF THE INVENTION

The present invention is directed to a method for treating neoplastic, angiogenic, fibroblastic, and/or immunosuppressive ocular irregularities of a living subject comprising the steps of: (a) providing a living subject, wherein the living subject includes an affected ocular area having a neoplastic, angiogenic, fibroblastic, and/or immunosuppressive irregularity; (b) providing a 5′ fluorouracil based medicament, wherein the 5′ fluorouracil based medicament is capable of inhibiting DNA synthesis; (c) associating a therapeutically effective concentration of the 5′ fluorouracil based medicament with the affected ocular area of the living subject; and (d) decreasing the neoplastic, angiogenic, fibroblastic, and/or immunosuppressive ocular irregularity of the living subject.

In a preferred embodiment of the present invention, wherein the step of providing a 5′ fluorouracil based medicament includes the step of providing a medicament represented by the following chemical structure:

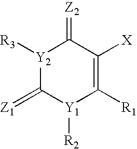

wherein R₁₋₃ are the same or different and comprise H, NH₂, a hydroxy group, a straight or branched alkyl, cycloalkyl, polycycloalkyl, heterocycloalkyl, aryl, alkaryl, aralkyl, alkoxy, alkenyl, alkynyl group containing approximately 1 to approximately 25 carbon atom(s), a silyl or siloxyl group containing approximately 1 to approximately 25 silicon atom(s), and combinations thereof, wherein X comprises F, Cl, Br, I, At, and/or any −1 monoatomic or polyatomic anion; wherein Y₁₋₂ comprises N or P; and wherein Z₁₋₂ comprises O or S. In this embodiment of the present invention the step of providing a fluoro5′ fluorouracil based medicament includes the step of providing a medicament represented by the following chemical structure:

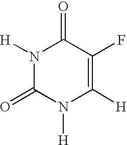

In yet another preferred embodiment of the present invention, the step of associating a therapeutically effective concentration of the 5′ fluorouracil based medicament with the living subject includes the step of ocular iontophoretic delivery of the medicament in a concentration ranging from approximately 0.5 to approximately 50 mg/mL per day for approximately 1 to approximately 30 days.

The present invention is also directed to a method for treating an affected area of a living subject's eye, comprising the steps of: (a) associating a 5′ fluorouracil based medicament with an ocular iontophoretic device; (b) positioning at least a portion of the ocular iontophoretic device on the eye of a living subject; and (c) iontophoretically delivering the 5′ fluorouracil based medicament to an affected area of the living subject's eye.

In a preferred embodiment of the present invention, the step of associating the 5′ fluorouracil based medicament includes the step of associating a 5′ fluorouracil based medicament capable of decreasing neoplastic, angiogenic, fibroblastic, and/or immunosuppressive ocular irregularities of the living subject.

Preferably, the step of iontophoretically delivering the 5′ fluorouracil based medicament includes delivering the same to at least one of the group consisting of the sclera, ciliary body, iris, lens, cornea, aqueous fluid, vitreous body, retina, choroids, optic nerve, and regions of the eye thereabout.

In accordance with the present invention, the step of iontophoretically delivering the 5′ fluorouracil based medicament may include the step of iontophoretically delivering the 5′ fluorouracil medicament using a negative polarity current between approximately 0.5 mA and approximately 5 mA for a period of between approximately 1 and approximately 60 minutes.

The present invention is further directed to an ocular iontophoretic device for delivering a 5′ fluorouracil based medicament to an affected area of a living subject's eye, comprising an active electrode assembly associated with a matrix, wherein the matrix includes a 5′ fluorouracil based medicament capable of decreasing neoplastic, angiogenic, fibroblastic, and/or immunosuppressive ocular irregularities of the living subject.

In a preferred embodiment of the present invention, the ocular iontophoretic device further comprises: (a) a counter electrode assembly, wherein the counter electrode assembly is configured for completing an electrical circuit between the active electrode assembly and an energy source; and (b) an energy source for generating an electrical potential difference.

In accordance with the present invention, the active electrode assembly may include an open-faced or high current density electrode.

The present invention is also directed to an ocular iontophoretic device for delivering a 5′ fluorouracil based medicament to an affected area of a living subject's eye, comprising: (a) a matrix, wherein the matrix is capable of temporarily retaining a solution having a 5′ fluorouracil based medicament capable of decreasing neoplastic, angiogenic, fibroblastic, and/or immunosuppressive ocular irregularities of the living subject; (b) an active electrode assembly associated with the matrix, wherein the active electrode assembly is configured for iontophoretically delivering the 5′ fluorouracil based medicament to the affected area of the living subject's eye; (c) a counter electrode assembly, wherein the counter electrode assembly is configured for completing an electrical circuit between the active electrode assembly and an energy source; and (d) an energy source for generating an electrical potential difference.

The present invention further includes an ocular iontophoretic device for delivering a 5′ fluorouracil based medicament to an affected area of a living subject's eye, comprising: (a) a reservoir, wherein the reservoir includes a 5′ fluorouracil based medicament capable of decreasing neoplastic, angiogenic, fibroblastic, and/or immunosuppressive ocular irregularities of the living subject; (b) a matrix, wherein the matrix is capable of temporarily retaining a solution having a 5′ fluorouracil based medicament; (c) an active electrode assembly associated with the matrix, wherein the active electrode assembly is configured for iontophoretically delivering the 5′ fluorouracil based medicament to the affected area of the living subject's eye; (d) a counter electrode assembly, wherein the counter electrode assembly is configured for completing an electrical circuit between the active electrode assembly and an energy source; and (e) an energy source for generating an electrical potential difference.

### BRIEF DESCRIPTION OF THE DRAWINGS

The invention will now be described with reference to the drawings wherein:

FIG. 1 of the drawings is a cross-sectional schematic representation of a first embodiment of an ocular iontophoretic device fabricated in accordance with the present invention;

FIG. 2 of the drawings is a cross-sectional schematic representation of a first embodiment of an ocular iontophoretic device fabricated in accordance with the present invention showing the association of a counter electrode assembly and an energy source; and

FIG. 3 of the drawings is a cross-sectional schematic representation of a second embodiment of an ocular iontophoretic device fabricated in accordance with the present invention.

### DETAILED DESCRIPTION OF THE INVENTION

While this invention is susceptible of embodiment in many different forms, there is shown in the drawings and will herein be described in detail several specific embodiments with the understanding that the present disclosure is to be considered as an exemplification of the principles of the invention and is not intended to limit the invention to the embodiments illustrated.

It will be understood that like or analogous elements and/or components, referred to herein, may be identified throughout the drawings with like reference characters.

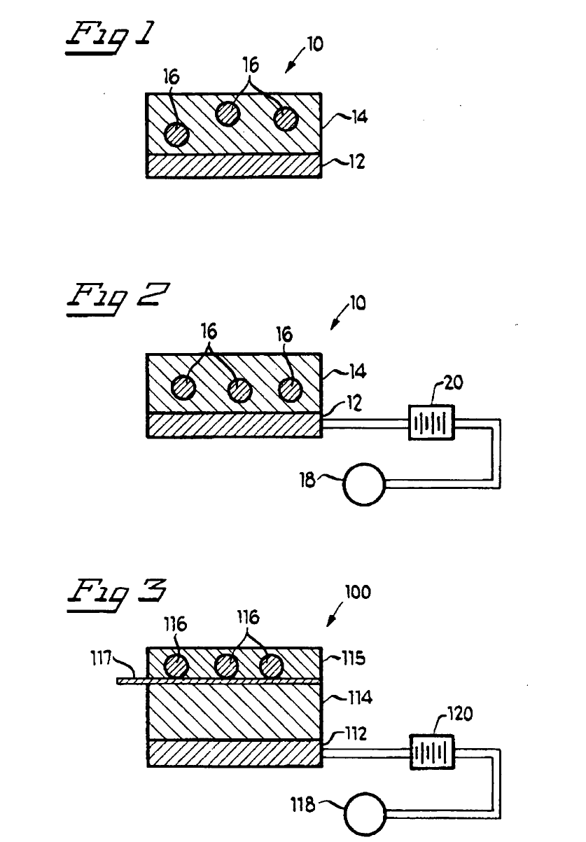

Referring now to the drawings and to FIG. 1 in particular, a first embodiment of an ocular iontophoretic device 10 is shown, which generally comprises active electrode assembly 12 and matrix 14. It will be understood that FIG. 1 is merely a cross-sectional schematic representation of ocular iontophoretic device 10. As such, some of the components have been distorted from their actual scale for pictorial clarity. As will be discussed in greater detail below, ocular iontophoretic device 10 is configured for delivering one or more 5′ fluorouracil based medicament(s) which are capable of acting as an inhibitor of DNA, and, therefore, treating, among other things, neoplastic, angiogenic, fibroblastic, and/or immunosuppressive ocular irregularities. By iontophoretically administering a 5′ fluorouracil based medicament to an affected area of a living subject's eye, diseases associated with the above-identified ocular irregularities can be efficiently remedied—especially including diseases of the eye wherein the affected area is toward the back of the eye, or generally proximate the optic nerve. Moreover, by utilizing iontophoretic technology, the living subject does not need to be exposed to such high medicament concentrations, which is of particular importance with such a potent classification of medicaments, because toxicity build can occur rapidly using conventional, for example, systemic administration methods. Ocular iontophoretic device 10 offers many advantages over the previously discussed prior art devices and associated delivery methods, including, but not limited to, simultaneous enablement of non-invasive and deep 5′ fluorouracil based medicament delivery, non-invasive local delivery of an effective, therapeutic level of 5′ fluorouracil based medicament while minimizing systemic concentrations, and enablement of, for example, sclera loading for prolonged delivery (of controlled, sometimes, low concentrations of medicaments) into regions in the back of the eye.

Active electrode assembly 12 generally comprises a conductive material, which upon application of an electrical potential difference thereto, drives an ionic 5′ fluorouracil based medicament (i.e. an anionic medicament), received from matrix 14 and delivers the 5′ fluorouracil based medicament into predetermined tissues and surrounding structures of a living subject's eye. It will be understood that active electrode assembly 12 may comprise an anode or a cathode depending upon whether the medicament is cationic or anionic in form. It will be further understood that active electrode assembly may include an open-faced or high current density electrode. As would be readily understood to those having ordinary skill in the art, any one of a number of conventional active electrode assemblies are contemplated for use in accordance with the present invention. The only contemplated limitation relative to active electrode assembly 12 is that it must be geometrically and compositionally compatible for ocular applications of living subjects, most relevantly, humans.

Matrix 14 extends contiguously from active electrode 12, and is preferably fabricated from a material capable of temporarily retaining 5′ fluorouracil based medicament 16 in solution. The solution may also contain supplemental agents, such as electrolytes, stability additives, medicament preserving additives, pH regulating buffers, etc. Matrix 14 may comprise, for example, a natural or synthetic amorphous member, a natural or synthetic sponge pad, a natural or synthetic lint free pad, a natural or synthetic low particulate member—just to name a few. Indeed, numerous other materials that would be known to those having ordinary skill in the art having the present disclosure before them are likewise contemplated for use. As with active electrode assembly 12, the only contemplated limitation relative to matrix 14 is that it must be geometrically and compositionally compatible for ocular applications of living beings, most relevantly, humans.

Medicament 16 is retained within matrix 14. In accordance with the present invention, ionic medicament 16 comprises one or more 5′ fluorouracil based medicament(s) which are capable of treating, among other things, neoplastic, angiogenic, fibroblastic, and/or immunosuppressive ocular irregularities.

Such 5′ fluorouracil based medicaments may be represented by the following chemical structure:

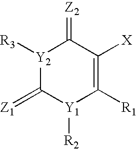

wherein R₁₋₃ are the same or different and comprise H, NH₂, a hydroxy group, a straight or branched alkyl, cycloalkyl, polycycloalkyl, heterocycloalkyl, aryl, alkaryl, aralkyl, alkoxy, alkenyl, alkynyl group containing approximately 1 to approximately 25 carbon atom(s), a silyl or siloxyl group containing approximately 1 to approximately 25 silicon atom(s), and combinations thereof; and the pharmaceutically acceptable acid addition salts thereof. It will be understood that the availability of 5′ fluorouracil medicaments will be readily known to those having ordinary skill in the art, and that derivatives thereof may be obtained using conventional organic synthetic routes.

For example, the 5′ fluorouracil based medicament may comprise the chemical structure:

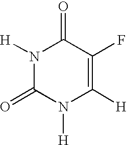

As is shown in FIG. 2, ocular iontophoretic device 10 may also include counter electrode assembly 18 and energy source 20. Counter electrode assembly 18 may be housed within ocular iontophoretic device 10, or alternatively, may be remotely associated with ocular iontophoretic device 10 via conventional electrical conduit. Counter electrode assembly 18 is configured for completing an electrical circuit between active electrode assembly 12 and energy source 20. As with active electrode 12, counter electrode 18 may comprise an anode or a cathode depending upon whether the medicament is cationic or anionic in form. As would be readily understood to those having ordinary skill in the art, any one of a number of counter electrodes are contemplated for use in accordance with the present invention.

Similarly to counter electrode assembly 18, energy source 20 may be housed within ocular iontophoretic device 10, or alternatively, may be remotely associated with ocular iontophoretic device 10 via conventional electrical conduit. Energy source 20 preferably supplies low voltage constant direct current between approximately 0.5 milliamps (mA) and approximately 5 mA for generating an electrical potential difference. The energy source may also provide for an initial higher voltage during current ramp-up to break down higher initial tissue resistance as in commercial power supply units used for transdermal iontophoresis. For purposes of the present disclosure, energy source 20 may include one or more primary or secondary electrochemical cells. While specific examples of energy source 20 have been disclosed, for illustrative purposes only, it will be understood that other energy sources known to those having ordinary skill in the art having the present disclosure before them are likewise contemplated for use.

Referring now to the drawings and to FIG. 3 in particular, a second embodiment of an ocular iontophoretic device 100 is shown, which generally comprises active electrode assembly 112, matrix 114, reservoir 115, counter electrode assembly 118, and energy source 120. It will be understood that active electrode assembly 112, matrix 114, counter electrode assembly 118, and energy source 120, are configured analogously to previously discussed active electrode assembly 12, matrix 14, counter electrode assembly 18, and energy source 20, respectively. Ocular iontophoretic device 100 is configured for delivering a 5′ fluorouracil based medicament to an affected area of a living subject's eye for treating neoplastic, angiogenic, fibroblastic, and/or immunosuppressive ocular irregularities.

Reservoir 115 includes 5′ fluorouracil based medicament 116, in solution, which is capable of treating the above-identified ocular irregularities. Reservoir 115 may include a releasable cover member 117 which, upon articulation, releases 5′ fluorouracil based medicament 116 into matrix 114. Such a release cover enables prompt delivery of the −5′ fluorouracil based medicament with very little device preparation.

The present invention is also directed to a method for treating an affected area of a living subject's eye comprising the following steps. First, a 5′ fluorouracil based medicament is associated with an ocular iontophoretic device. Preferably, the 5′ fluorouracil based medicament is metered from a syringe or single unit dose. Second, at least a portion of the ocular iontophoretic device is positioned on the eye of a living being. Finally, the 5′ fluorouracil based medicament is iontophoretically delivered to an affected area of the living subject's eye. Preferably, the delivery lasts for between approximately 1 and approximately 60 minutes. Compared to prior art administration or delivery methods, the present invention enables a generally painless, non-invasive, and deep delivery of the 5′ fluorouracil based medicament. Moreover, the 5′ fluorouracil based medicament is locally delivered to an affected area of a living subject's eye at an effective, therapeutic level. Preferred ocular delivery regions include the sclera, ciliary body, iris, lens, cornea, aqueous fluid, vitreous body, retina, choroids, optic nerve, and regions of the eye thereabout.

For purposes of the present disclosure, neoplastic, angiogenic, fibroblastic, and/or immunosuppressive ocular irregularities of a living subject can also be treated in accordance with the following method. First, a living subject with a neoplastic, angiogenic, fibroblastic, and/or immunosuppressive irregularity is provided. Second, one or more of the above-identified 5′ fluorouracil based medicaments is provided. Third, a therapeutically effective concentration of the 5′ fluorouracil based medicament is associated with and/or administered to the affected ocular area of the living subject. Preferably, the 5′ fluorouracil based medicament is administered in a concentration ranging from approximately 0.5 to approximately 50 mg/mL. The duration of a single application may range from 1 minute to approximately 60 minutes. The medicament may be administered on a schedule ranging from once every day to once every 30 days. The duration of 5′ fluorouracil based therapy may range from a single application to multiple applications that are administered over a period of months to years, depending upon the disease being treated. Upon administration of the m-5′ fluorouracil based medicament, the neoplastic, angiogenic, fibroblastic, and/or immunosuppressive ocular irregularity of the living subject is materially decreased.

It will be understood that while iontophoresis has been disclosed as one suitable means for the local ocular administration of 5′ fluorouracil based medicaments, any one of a number of other local administering means are likewise contemplated for use, such as via needle injection and/or topical administration with a pad.

5′ fluorouracil is dissolved in a balanced saline solution, for example, sodium chloride (e.g. 0.25 to 0.9% w/v). The solution may be buffered with other salts, such as phosphate. carbonate, or citrate. The pH is adjusted to a value between 4.0 and 9.0, preferably pH 7.5, using NaOH or HCl. The final concentration of 5′ fluorouracil is between 0.5 and 50 mg/mL. Iontophoretic current is applied at 1.0 to 4.0 milliamperes for 1 to 60 minutes. It will be understood to those having ordinary skill in the art that the previously identified formulation, although being preferred, is not the only formulation which can be used.

The foregoing description merely explains and illustrates the invention and the invention is not limited thereto except insofar as the appended claims are so limited, as those skilled in the art who have the disclosure before them will be able to make modifications without departing the scope of the invention.

What is claimed is:

1. A method for treating neoplastic, angiogenic, fibroblastic, and/or immunosuppressive ocular irregularities of a living subject, comprising the steps of:
providing a living subject, wherein the living subject includes an affected ocular area having a neoplastic, angiogenic, fibroblastic, and/or immunosuppressive irregularity;
providing a uracil based medicament, wherein the uracil based medicament is capable of inhibiting DNA synthesis;
associating a therapeutically effective concentration of the uracil based medicament with the affected ocular area of the living subject; and
decreasing the neoplastic, angiogenic, fibroblastic, and/or immunosuppressive ocular irregularity of the living subject.

2. The method according to claim 1, wherein the step of providing a uracil based medicament includes the step of providing a medicament represented by the following chemical structure: 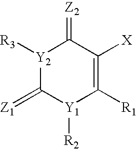
wherein R₁₋₃ are the same or different and comprise H, NH₂, a hydroxy group, a straight or branched alkyl, cycloalkyl, polycycloalkyl, heterocycloalkyl, aryl, alkaryl, aralkyl, alkoxy, alkenyl, alkynyl group containing approximately 1 to approximately 25 carbon atom(s), a silyl or siloxyl group containing approximately 1 to approximately 25 silicon atom(s), and combinations thereof;
wherein X comprises F, Cl, Br, I, At, and/or any −1 monoatomic or polyatomic anion;
wherein Y₁₋₂ comprises N or P; and
wherein Z₁₋₂ comprises O or S.

3. The method according to claim 1, wherein the step of providing a uracil based medicament includes the step of providing a medicament represented by the following chemical structure:

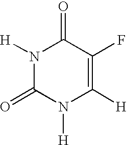

4. The method according to claim 1, wherein the step of providing a uracil based medicament includes the step of providing 5-Fluoro-1-H-pyrimidine-2,4-dione and derivatives thereof.

5. The method according to claim 1, wherein the step of providing a uracil based medicament includes the step of providing 5′ fluorouracil.

6. The method according to claim 1, wherein the step of associating a therapeutically effective concentration of the uracil based medicament with the living subject includes the step of ocular iontophoretic delivery of the medicament in a concentration ranging from approximately 0.5 to approximately 50 mg/mL per day for approximately 1 to approximately 30 days.

7. A method for treating an affected area of a living subject's eye, comprising the steps of:
associating a uracil based medicament with an ocular iontophoretic device;
positioning at least a portion of the ocular iontophoretic device on the eye of a living subject; and
iontophoretically delivering the uracil based medicament to an affected area of the living subject's eye.

8. The method according to claim 7, wherein the step of associating the uracil based medicament includes the step of associating a uracil based medicament capable of decreasing neoplastic, angiogenic, fibroblastic, and/or immunosuppressive ocular irregularities of the living subject.

9. The method according to claim 7, wherein the step of iontophoretically delivering the uracil based medicament includes the step of iontophoretically delivering the uracil based medicament to at least one of the group consisting of the sclera, ciliary body, iris, lens, cornea, aqueous fluid, vitreous body, retina, choroids, optic nerve, and regions of the eye thereabout.

10. The method according to claim 7, wherein the step of iontophoretically delivering the uracil based medicament includes the step of iontophoretically delivering the uracil medicament at a current between approximately 0.5 mA and approximately 5 mA for a period of between approximately 1 and approximately 60 minutes.

11. The method according to claim 7, wherein the step of iontophoretically delivering the uracil based medicament includes the step of delivering the uracil based medicament using negative polarity electrical current.

12. A method for achieving an effect in a living subject, comprising:
administering an effective amount of a uracil based medicament to the living subject, wherein the effect is decreasing a neoplastic, angiogenic, fibroblastic, and/or immunosuppressive ocular irregularity of the living subject.

13. The method of claim 12, wherein the step of administering the effective amount of the uracil based medicament comprises the step of utilizing a compound selected from the group consisting of 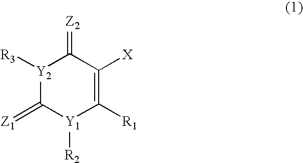
wherein R₁₋₃ are the same or different and comprise H, NH₂, a hydroxy group, a straight or branched alkyl, cycloalkyl, polycycloalkyl, heterocycloalkyl, aryl, alkaryl, aralkyl, alkoxy, alkenyl, alkynyl group containing approximately 1 to approximately 25 carbon atom(s), a silyl or siloxyl group containing approximately 1 to approximately 25 silicon atom(s), and combinations thereof;
wherein X comprises F, Cl, Br, I, At, and/or any −1 monoatomic or polyatomic anion;
wherein Y₁₋₂ comprises N or P; and,
wherein Z₁₋₂ comprises O or S; 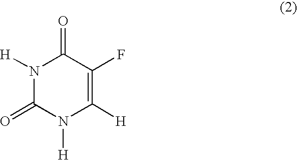
(3) 5-Fluoro-1-H-pyrimidine-2,4-dione and derivatives thereof;
(4) 5′ fluorouracil; and,
(5) any combination thereof, to the living subject, wherein the effect is decreasing a neoplastic, angiogenic, fibroblastic, and/or immunosuppressive ocular irregularity of the living subject.

14. The method of claim 13, wherein the uracil based medicament is formulated in an approximately 0.5 mg/mL compound and approximately 50 mg/mL compound buffer.

15. The method of claim 13, wherein the buffer ranges in pH from approximately 4.0 to approximately 9.0.

16. The method of claim 15, wherein the buffer is pH 7.5.
